(19) United States
(12) Patent Application Publication
(10) Pub. No.: US 2017/0296460 A1
(43) Pub. Date: Oct. 19, 2017

(54) METHOD OF IMPROVING THE APPEARANCE OF PERIORBITAL DYSCHROMIA

(71) Applicant: The Procter & Gamble Company, Cincinnati, OH (US)

(72) Inventor: Karen Marie OSORIO, CIncinnati, OH (US)

(21) Appl. No.: 15/099,087

(22) Filed: Apr. 14, 2016

(51) Int. Cl. A61K 8/97; A61K 8/06; A61B 5/103; A61B 5/00; A61Q 19/00

(52) U.S. Cl. CPC A61K 8/97; A61B 5/444; A61Q 19/00; A61B 5/1032; A61K 8/062; A61K 2800/74; A61K 2800/48

(57) ABSTRACT

A method of improving the appearance of periorbital dyschromia by identifying a target portion of skin in the periorbital region of a person that exhibits periorbital dyschromia and applying a personal care composition to the target portion of skin. The personal care composition includes an effective amount of an extract of Cucurbita pepo and a dermatologically acceptable carrier. The personal care composition is applied for a sufficient amount of time for the extract of Cucurbita pepo to improve the appearance of the periorbital dyschromia.

### FIELD

The present disclosure is directed generally to a method of improving the appearance of periorbital dyschromia. More specifically, the present disclosure is directed to improving the appearance of one or more types of periorbital dyschromia by applying a cosmetic composition comprising an extract of Cucurbita pepo to periorbital skin in need of such treatment.

### BACKGROUND

Periorbital dyschromia, which is sometimes referred to as under-eye dark circles, is generally recognized as an undesirable discoloration of the skin around the eyes, and is commonly associated with fatigue and/or aging. A variety of ways to improve the appearance of periorbital dyschromia have been devised, such as applying concealers and/or other cosmetic products to hide its appearance. But using makeup to hide the appearance of periorbital dyschromia is only a temporary solution. In order to maintain the cosmetic benefit provided by conventional makeup products, a user will typically apply the product daily and, in some instances, may even be required to reapply it throughout the day. Thus, a more permanent solution is desired to reduce and/or eliminate some of the undesirable aesthetic features commonly found around the eye, for example, by addressing the underlying causes(s) of the periorbital dyschromia.

In an effort to find a solution to the problem of periorbital dyschromia, researchers have tried to identify the underlying causes of the condition. Currently, periorbital dyschromia is recognized as a multifactorial pathogenesis that is not well elucidated. While it is generally known that there may be different types of periorbital dyschromia, there is no universally recognized definition for each type. And even among those researchers who recognize that there are different types of periorbital dyschromia, some still propose treating different types of periorbital dyschromia with a single composition or material in a “one size fits all” approach. Thus, there remains a need for a method of treating periorbital dyschromia that is tailored to separately address the characteristics associated with a particular type of periorbital dyschromia.

Accordingly, it would be desirable to provide a method of treating periorbital dyschromia in which a chronic active is applied to a target portion of skin that exhibits periorbital dyschromia to improve the appearance of the periorbital dyschromia. It would also be desirable to provide a method of treating periorbital dyschromia by applying a cosmetic composition comprising an extract of Cucurbita pepo to a target portion of skin that exhibits periorbital dyschromia to improve the appearance of the periorbital dyschromia. It would further be desirable to provide a method of treating periorbital dyschromia by applying a cosmetic composition comprising an extract of Cucurbita pepo to a target portion of skin to improve the appearance of a particular type of periorbital dyschromia.

### SUMMARY

The method disclosed herein provides a solution to the problem of improving the appearance of Type II and Type III periorbital dyschromia. The method comprises identifying a target portion of periorbital skin exhibiting Type II or Type III periorbital dyschromia and applying a personal care composition to the target portion of skin. The personal care composition comprises an effective amount of an extract of Cucurbita pepo, a dermatologically acceptable carrier, and a viscosity of between 50,000 and 200,000 cps. The improvement in the appearance can correspond to a positive change in visual perception scale score, a decrease in blood perfusion, an increase in L* value, a decrease in a* value and/or an increase in b* value. In some instances, the composition includes spherical particles.

In some instances, the method of improving the appearance of periorbital dyschromia comprises identifying a target portion of periorbital skin exhibiting periorbital dyschromia and applying a first personal care composition to the target portion of skin. The first personal care composition comprises from about 0.0001% to about 15% by weight of the composition of an extract of Cucurbita pepo and a dermatologically acceptable carrier. The method further comprises applying a second personal care composition to the target portion of skin. The second personal care composition comprises a skin care agent and a dermatologically acceptable carrier.

### BRIEF DESCRIPTION OF THE DRAWINGS

FIG. 1 is an illustration of various portions of a human face.

FIGS. 2A and 2B illustrate examples of the portion of the periorbital region affected by Type I periorbital dyschromia.

FIGS. 3A and 3B illustrate examples of portion of the periorbital region affected by Type II periorbital dyschromia.

FIGS. 4A and 4B illustrate examples of portion of the periorbital region affected by Type III periorbital dyschromia.

### DETAILED DESCRIPTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are weight percentages based on the weight of the composition, unless specifically stated otherwise. All ratios are weight ratios, unless specifically stated otherwise. All ranges are inclusive and combinable, are inclusive of narrower ranges, and delineated upper and lower range limits are interchangeable to create further ranges not explicitly delineated. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity.

The compositions herein can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

“Chronic active” means an active suitable for use in a topical cosmetic composition that continues to provide the desired benefit after use of the active is discontinued. Chronic actives provide a relatively long lasting cosmetic benefit as compared to the acute actives commonly found in conventional makeup products that are intended to cover or hide perceived cosmetic flaws (e.g., the pigments, dyes, lakes and other colorants commonly found in foundations and concealers). In some instances, chronic actives work via recurrent use of the active over an extended period of time (e.g., use of the active for more than 1 week). In contrast, acute actives have no lasting effect on the skin, and once the acute active is removed, the skin is the same in appearance as before the acute active was applied. Compositions containing chronic actives may be applied on the order of about once per day over such extended periods. In some instances, the application rates may vary from about once per week to about three times per day or at some rate in between. The chronic active may provide the desired benefit almost immediately, or after some minimum amount of recurring use of the composition (e.g., after 1, 2, 3, 4, 5, 6, 7, 8, 9, 19, 11, or even 12 weeks). The benefit provided by the chronic active may last for more than 1 day (e.g., more than 2, 3, 4, 5, or 6 days), more than 1 week (e.g., more than 2, 3, or 4 weeks) or even more than a month after use of the composition containing the chronic active is discontinued.

“Cosmetic” means providing a desired visual effect on an area of the human body. The visual effect may be temporary, semi-permanent, or permanent. Some non-limiting examples of “cosmetic products” include products that leave color on the face, such as foundation, mascara, concealers, eye liners, brow colors, eye shadows, blushers, lip sticks, lip balms, face powders, solid emulsion compact, and the like.

“Cosmetic agent” means any substance, as well any component thereof, suitable for use in a topical cosmetic composition intended to be contacted with (e.g., rubbed, poured, sprinkled, sprayed, introduced into, or otherwise applied to) a mammalian body or any part thereof to provide a cosmetic effect. Cosmetic agents may be chronic or acute and may include substances that are Generally Recognized as Safe (GRAS) by the US Food and Drug Administration, food additives, and materials used in non-cosmetic consumer products including over-the-counter medications.

“Cosmetic composition” means any composition comprising a cosmetic agent that is suitable for topical application to mammalian skin.

“Disposed” refers to an element being located in a particular place or position relative to another element.

“Effective amount” means an amount of a compound or composition sufficient to significantly induce a positive appearance and/or feel benefit but low enough to avoid serious side effects, i.e., to provide a reasonable benefit to risk ratio, within the scope of sound judgment of the skilled artisan. In the present method, an effective amount of pumpkin seed extract is an amount sufficient to improve the appearance of Type II and/or Type III periorbital dyschromia during a treatment period.

“Improve the appearance of” means effecting a desirable change or benefit in periorbital dyschromia appearance. For example, an improvement in the appearance of Type II or Type III periorbital dyschromia can correspond to a positive score on the Visual Perception Scale (“VPS”); a decrease in blood perfusion; an increase in L* value; a decrease in a* value and/or an increase in b* value.

“L*a*b*” refers to the commonly recognized color space specified by the International Commission on Illumination (“CIE”). The three coordinates represent the lightness of the color (L*=0 yields black and L*=100 indicates diffuse white), its position between magenta and green (a*, negative values indicate green while positive values indicate magenta) and its position between yellow and blue (b*, negative values indicate blue and positive values indicate yellow).

“Periorbital” means around the orbit of the eye. The periorbital region of a person is the area of the face generally disposed around the eye socket and typically lies between the bottom of the brow and the top of the cheek in the longitudinal direction and between the bridge of the nose and the temple in the lateral direction.

“Periorbital Dyschromia” is a condition that occurs when the tone of the skin in the periorbital region of person appears noticeably different from tone of the skin in a nearby portion of the face such as the cheek, nose, forehead, temple and/or another portion of the periorbital region. Periorbital dyschromia is generally bilateral (i.e., it occurs in the periorbital region of both sides of the face). Periorbital dyschromia may manifest as the appearance of a difference in skin tone in the periorbital region relative to other regions of the face and/or body (e.g., cheek, nose, forehead, temple, chin). Periorbital dyschromia may appear as a result of hyperpigmented or hypopigmented skin in the periorbital region. In some instances, periorbital dyschromia may be classified visually by an expert grader (i.e., someone trained to visually classify periorbital dyschromia) either in-person or from a captured image. In some instances, periorbital dyschromia may be classified visually by an expert grader (i.e., someone trained to visually classify periorbital dyschromia) either in-person or from a captured image. In some instances, periorbital dyschromia may be analyzed and/or classified using a diagnostic device configured to use an imaging technique. It may be desirable to place such diagnostic devices and/or expert graders in a retail environment, for example near cosmetic eye-care products. Type I, Type II and Type III periorbital dyschromia are described in more detail below.

“Personal care composition” means a composition suitable for topical application on mammalian keratinous tissue that provides an acute or chronic benefit to the keratinous tissue or a type of cell commonly found therein.

“Topical application” means to apply or spread the compositions of the present invention onto the surface of the keratinous tissue.

The discovery that there are different types of periorbital dyschromia with different underlying biological causes and appearances has led to a need to identify chronic actives and/or combinations of actives that can provide tailored treatment solutions for treating each of the different types of periorbital dyschromia. It has surprisingly been found that an extract of Cucurbita pepo, which is more commonly known as pumpkin, can improve the appearance of periorbital dyschromia, and in particular Type II and/or Type III periorbital dyschromia. Extracts of pumpkin seed may be especially desirable for use herein. While pumpkin seed extract (INCI name: Cucurbita pepo Seed Extract; CAS No. 289-741-0) is known for use in topical skin care compositions for promoting general skin health and as a 5-α-reductase inhibitor (see, e.g., U.S. Pat. No. 8,048,456 and U.S. Pub. No. 2013/0309217), it was not previously known that pumpkin seed extract improves the appearance of Type II and Type III periorbital dyschromia. In addition, the present studies also suggest that pumpkin seed extract does not adversely affect the appearance of Type I periorbital dyschromia, which is especially desirable in the event a consumer's periorbital dyschromia type is misidentified and pumpkin seed extract is used to treat it.

### Types of Periorbital Dyschromia

There are a variety of evaluation techniques suitable for identifying and/or evaluating the type of periorbital dyschromia exhibited by person (e.g., visual evaluation, blood perfusion, image analysis, histological analysis, biomarker analysis, gene expression signature analysis and/or gene expression theme analysis). Suitable examples of systems and methods of classifying periorbital dyschromia and descriptions of the different types are described in U.S. Ser. No. 14/215,785, filed on Mar. 17, 2014 by Osorio, et al., and titled “Methods of Classifying Periorbital Dyschromia and Systems Therefor.” In the present method, the periorbital dyschromia exhibited by a person may be classified as Type I, Type II, or Type III. Alternatively, a person may have a “No Dyschromia” condition.

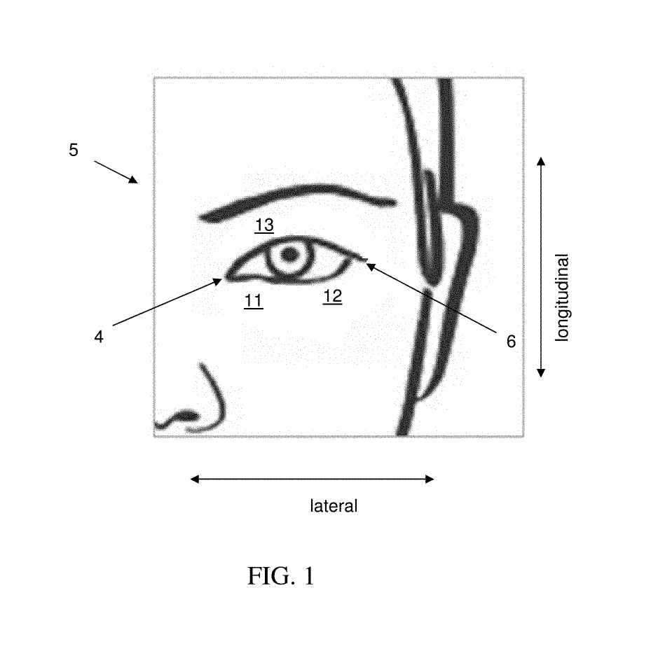

FIG. 1 illustrates the periorbital region of a human face 5 divided into three zones 11, 12 and 13, which are useful for helping identify the different types periorbital dyschromia. Zone 1 11 is disposed generally in the inner portion of the under-eye area and extends laterally from the inner corner 4 of the eye to about half the distance to the outer corner 6 of the eye. Zone 2 12 extends from the distal edge of Zone 1 11 (i.e., from about the midpoint under the eye) to the outer corner 6 of the eye. Zone 1 11 and Zone 2 12 extend longitudinally from the lower eyelid to the top of the cheekbone. Zone 3 is disposed above the eye and extends laterally from the inner corner 4 of the eye to the outer corner 6 of the eye. Zone 3 13 also extends longitudinally from the top of the eye to the eyebrow.

Type I periorbital dyschromia is visually characterized by continuous discoloration of both the upper and lower eyelid skin. The discolored periorbital skin associated with Type I periorbital dyschromia typically includes substantially uniform brown, yellow and/or orange tones in the skin of the periorbital region, which may resemble the color of tanned skin or an age spot. Type I periorbital dyschromia may also be generally defined, in part, by its location in the upper and lower portions of the periorbital regions (i.e., proximate the lower eyelid and the upper eyelid). In other words, Type I periorbital dyschromia is typically exhibited in Zones 1 and 3 and, in some instances, Zone 2. Type II periorbital dyschromia is characterized by continuous discoloration of the lower eyelid skin. The discolored periorbital skin associated with Type II periorbital dyschromia typically includes substantially uniform purple, pink and/or bluish tones, which may resemble the color of bruised skin. Type II is generally defined, in part, by its presence in the inner, lower portion of the periorbital region (i.e., Zone 1) and its absence in the upper portion of the periorbital region (i.e., upper eyelid or Zone 3) and, in some instances, the outer, lower portion (i.e., Zone 2). Type III periorbital dyschromia is characterized by the presence of skin tones that resemble sunburned skin. Type III is generally defined, in part, by its presence in the under-eye and above-the-eye portions of the periorbital region. A No Dyschromia condition may be visually characterized by the lack of an uneven or discontinuous skin tone in the periorbital region.

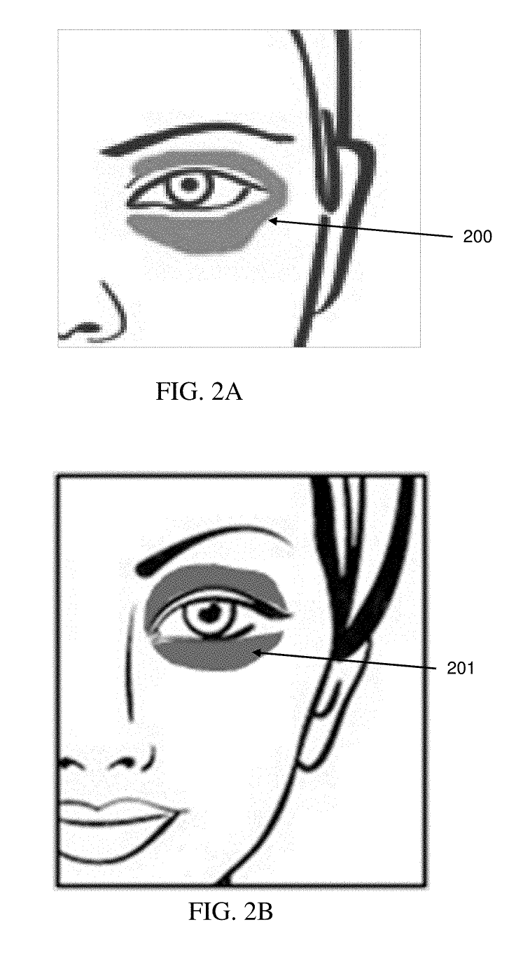

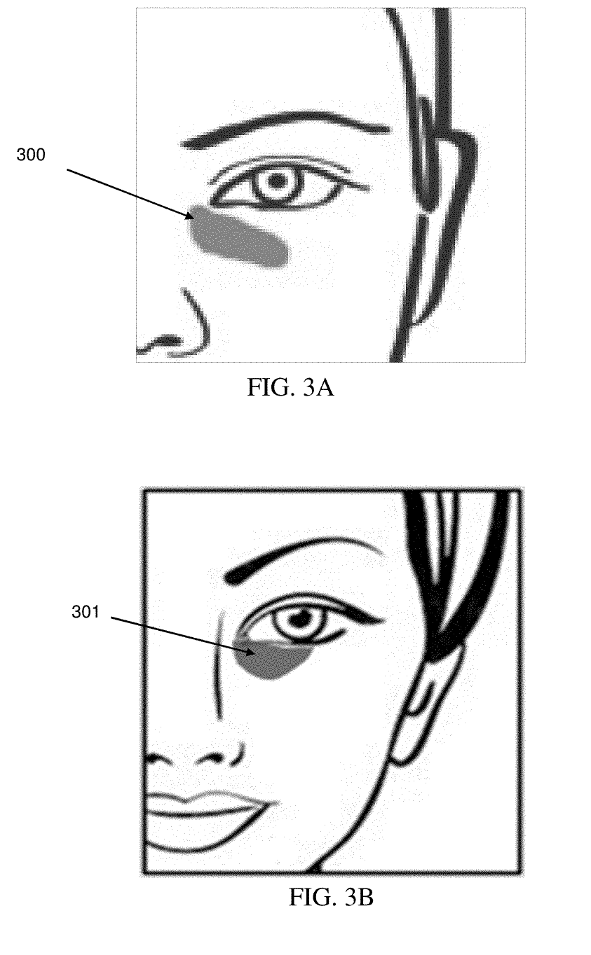

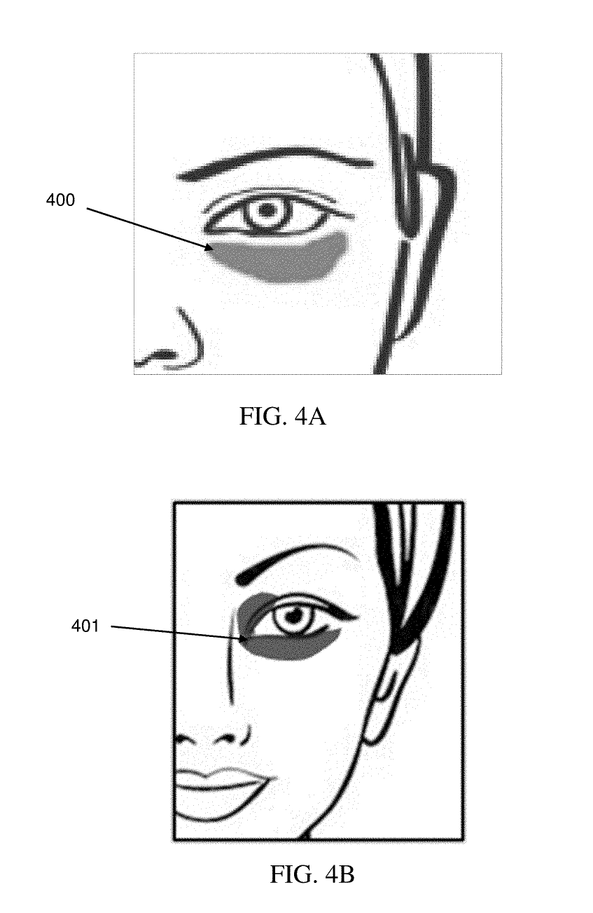

FIGS. 2A and 2B illustrate examples of Type I periorbital dyschromia, which is represented by the shaded portions 200 and 201, respectively, of the periorbital region. FIGS. 3A and 3B illustrate examples of Type II periorbital dyschromia (i.e., the shaded portions 300 and 301, respectively, of the periorbital region). FIGS. 4A and 4B illustrate examples of Type III periorbital dyschromia (i.e., the shaded portions 400 and 401, respectively, of the periorbital region). In some instances, the type of periorbital dyschromia may be identified according to the present method based on its location in the periorbital region, as illustrated in FIGS. 2A, 2B, 3A, 3B, 4A and/or 4B.

Different types of periorbital dyschromia may be distinguished from one another using known imaging techniques such as RGB color imaging. For example, Type I periorbital dyschromia may be characterized by generally having lower RGB values relative to Types II and III. Type II periorbital dyschromia may be characterized by generally having higher RGB values compared to Types I and III. Type III periorbital dyschromia may include characteristics of both Type I and Type II.

Type I, Type II and Type III periorbital dyschromia may also be distinguished from one another using histological evaluation techniques that include, for example, sectioning and staining, followed by examination under a microscope (e.g., light or electron). In particular, it has been found that the abundance and/or location of certain cellular structures (e.g., melanin) within skin biopsy samples obtained from periorbital skin may be used to distinguish Type I, Type II and Type III periorbital dyschromia from one another. For example, Type I periorbital dyschromia may be characterized by an over-abundance of melanin in the epidermis and the unexpected presence of melanin in the dermis of a skin sample. On the other hand, Type II periorbital dyschromia may characterized by an unexpected scarcity of melanin in the epidermis and an absence of melanin in the dermis. Type III periorbital dyschromia may be characterized by a combination of Type I and Type II characteristics.

In some instances, Type I, Type II and Type III periorbital dyschromia may be distinguished from one another by the scarcity and/or abundance of certain molecules in the epidermis of periorbital skin, such as pyrrole-2,3,5-tricarboxylic acid (“PTCA”), which is formed as a result of oxidative degradation of eumelanin. It has been found that Type I and Type III periorbital dyschromia have higher PTCA levels than Type II, and that Type I may exhibit higher PTCA levels than Type III.

The method herein includes the topical application of a personal care composition to a target skin surface exhibiting periorbital dyschromia. The personal care composition includes a safe and effective amount of an extract of Cucurbita pepo, i.e., an amount of Cucurbita pepo extract sufficient to improve the appearance of periorbital dyschromia, especially Type II and/or Type III, after a suitable course of treatment (e.g., at least 2, 4 or 8 weeks).

The target skin surface may be identified by the person exhibiting the periorbital dyschromia (e.g., via a self-assessment), an expert grader (e.g., in-person or from an image of the person), a diagnostic device in combination with a suitable diagnostic method (e.g., a digital camera in combination with suitable image analysis software), or a combination of these. For example, the method of identification may include examination of Zones 1, 2 and/or 3 of the periorbital region of a subject person and evaluating the color, location and/or intensity of the periorbital dyschromia to identify the periorbital dyschromia as a particular type (e.g., Type II or Type III). Once the periorbital dyschromia present on the target skin surface is identified, the present composition may be applied to the target skin surface for a period of time sufficient to improve the appearance of the periorbital dyschromia. Improvements in periorbital dyschromia herein can be demonstrated by a positive VPS score (e.g., from +0.1 to +4 or any value in this range), a decrease in blood perfusion (e.g., a decrease of at least 10, 12, 14, 16, 20 or even 25); an increase in L* value (e.g., an increase of at least 0.1, 0.2, 0.3, 0.4, 0.5, 0.6, 0.7, 0.8, 0.9 or even an increase of 1 or more); a decrease in a* value (e.g., a decrease of at least 0.1, 0.2, 0.3, 0.4, 0.5, 0.6, 0.7, 0.8, 0.9 or even a decrease of 1 or more); and/or an increase in b* value (e.g., an increase of at least 0.1, 0.2, 0.3, 0.4, 0.5, 0.6, 0.7, 0.8, 0.9 or even an increase of 1 or more). Methods for determining VPS score, blood perfusion and L*a*b* values are described in more detail below.

Type II and/or Type III periorbital dyschromia may be treated according to the present method by applying a composition comprising an effective amount of pumpkin seed extract to the dyschromic portion of skin about once a day, twice a day, or even more frequently during a treatment period. In some instances the composition may be applied one or more times per week but less than once per day, e.g., 2, 3, 4, 5, or 6 time per week. For example, the composition may be applied in the morning after showering, in the evening before bed and/or as part of a daily beauty regimen(s). The treatment period may last for 1 or more weeks (e.g., 2, 3, 4, 5, 6, 7 or even 8 weeks or more), multiple months (e.g., 2-12 months) or even multiple years. The treatment period should be long enough for the chronic active to improve the appearance of the periorbital dyschromia.

It may be desirable to apply the composition locally. As used herein, “localized”, “local”, and “locally” mean that the composition is delivered to the targeted area of skin (i.e., the target portion of periorbital skin exhibiting periorbital dyschromia) while minimizing delivery to portions of skin not requiring treatment. For example, the composition may be applied to the target skin surface in Zones 1, 2 and/or 3, depending on the type of periorbital dyschromia being treated, and lightly massaged into the target skin surface. Alternatively, the composition may be applied to the entire periorbital region or even the entire face. Alternatively, “general” or “generally”, when referring to applying the composition, means applying the composition to the target area of skin and one or more additional areas of skin other than the target area (e.g., a skin care composition that is applied to the entire face including a target portion of skin in the periorbital region).

In some instances, it may be desirable to use the present composition as part of a skin care regimen. For example, a first composition that includes an effective amount of Cucurbita pepo extract may be applied generally or locally to the skin in the periorbital regions of the face, and a second composition that includes one or more skin care agent (e.g., tone agents or moisturizers), may be applied to a portion of skin disposed outside the periorbital region (e.g., the entire face or a portion thereof). The first and second composition may be applied in any order, as desired, as long as the treatment effectiveness of the Cucurbita pepo extract is not undesirably inhibited.

The form of the composition or the dermatologically acceptable carrier should be selected to facilitate application. In some instances, the composition may be delivered with an applicator suitable for general and/or localized application. For example, the applicator may be configured to suitably apply from 1 to 50 μL/cm² of composition to a target skin surface. Of course, it will be appreciated that applicators are not required and the personal care composition herein can also be applied directly by using one's finger or in other conventional manners.

### Composition

Compositions suitable for use in the method herein include an effective amount of pumpkin seed extract disposed in a dermatologically acceptable carrier. The pumpkin seed extract herein may be a peptidic hydrolyzate resulting from the hydrolysis of the proteins of pumpkin seeds. The peptidic hydrolyzate generally includes a mixture of compounds predominantly represented by peptides. The term “peptide” refers to a sequence of two or more amino acids linked by peptide bonds or modified peptide bonds; whereas the term “polypeptide” designates a larger peptide (e.g. more than four). The use of peptidic hydrolyzates, in particular low molecular weight peptidic hydrolyzates, has many advantages in cosmetics. In addition to generating compounds of peptidic nature that did not already exist in the starting protein mixture, hydrolysis and purification make it possible to provide cosmetic compositions that are more stable, more easily standardizable, and causing fewer allergic reactions. An example of a pumpkin seed extract suitable for use herein is OCALINE PF, available from Soliance, France.

The pumpkin seed extract herein may be obtained by extracting proteins from the seeds of the Cucurbita pepo plant, hydrolyzing them and then, optionally, purifying the peptide fragments. Additionally or alternatively, the proteins may be extracted from the whole plant or a specific part of the plant (leaves, stems, roots, etc.). In some instances, the proteins are extracted by crushing the seeds (or other portions of the plant) and suspending the crushed seeds in an alkaline solution containing an insoluble polyvinylpolypyrrolidone (PVPP) adsorbent (0.01-20%), which facilitates the subsequent hydrolysis and purification operations. The soluble fraction, which contains the proteins and carbohydrates, is collected after centrifugation and filtering. This crude solution is then hydrolyzed under controlled conditions to generate soluble peptides. Hydrolysis is carried out chemically and/or advantageously with proteolytic enzymes. For the removal of polyphenol substances, an amount of PVPP can be added to the reaction medium in this controlled hydrolysis step. Next, the solution is filtered to eliminate the enzymes.

In the present treatment methods, pumpkin seed extract is included in a topical cosmetic composition as a chronic active for treating periorbital dyschromia, in particular Type II and/or Type III periorbital dyschromia. The pumpkin seed extract may be present at from 0.0001% to 15%, from 0.0002% to 10%, from 0.001% to 15%, from 0.025% to 10%, from 0.05% to 10%, from 0.05% to 5%, or even from 0.1% to 5%, by weight of the total composition. The amount of pumpkin seed extract that is “effective” can differ from one particular source (e.g., manufacturer) of extract to another, and can be determined by the skilled artisan based upon the particular extract product's level of activity (e.g., level of active components present). As with any extract, the concentration of active components in the particular extract product to be used will depend on factors such as the final dilution volume of the extract product, the particular extraction method employed, the natural range of variation among individual plants, and other common factors known to those skilled in the art.

The cosmetic compositions used in conjunction with the method herein may be in various product forms that include, but are not limited to, solutions, suspensions, lotions, creams, gels, toners, sticks, pencil, sprays, aerosols, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks (with and without an insoluble sheet), makeup such as foundations, eye liners, and eye shadows, and the like.

The skin in the periorbital region of a person is typically thinner and more delicate than the skin in many of other others of the face or body. Thus, it may be desirable for the present composition to have a viscosity that encourages regular use of the product. If the product viscosity is too low, it may be difficult to control the application of the product to the small, delicate eye area, as the product tends to spread or run too much on the skin and may even get into the eye, potentially causing irritation. On the other hand, if the viscosity is too high, the product may drag and pull on the skin as it is spread, making it difficult to apply or even damaging or irritating the delicate periorbital skin. Accordingly, products for use herein have a viscosity between 50,000 and 200,000 cps (e.g., between 70,000 and 150,000 cps, between 90,000 and 120,000 cps, or any value in these ranges). Viscosity is determined at 20° C.±2° C. using a BROOKFIELD DV-II+ brand viscometer or equivalent with a T-C spindle at 5 rpm with a heliopath setting.

In addition, due to its proximity to the eye, it may be desirable for the present compositions to have an opacity that provides an acute benefit and/or encourages regular use of the product. For example, if the opacity of the composition is too low, it may not conceal the appearance of the periorbital dyschromia it is intended to treat. On the other hand, in this example, if the product opacity is too high, the product may suitably conceal the appearance of periorbital dyschromia but result in a non-naturally looking appearance. The opacity of a composition may be determined according to the Contrast Ratio method described in more detail below. The compositions herein have a contrast ratio of from 5 to 40 (e.g., 7 to 30, or 8 to 20).

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g., water-soluble moisturizing agents, conditioning agents, anti-microbials, humectants and/or other skin care actives).

### Optional Ingredients

The present composition may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the periorbital dyschromia appearance improvement benefit provided by the present composition. When present, the additional ingredients may be included at amounts of from 0.0001% to 50%; from 0.001% to 20%; or even from 0.01% to 10%, by weight of the composition. The additional ingredients, when incorporated into the composition, should be suitable for use in contact with human skin tissue without undue toxicity, incompatibility, instability, allergic response, and the like. Some nonlimiting examples of additional ingredients which may be suitable for use herein are described in U.S. Publication Nos. 2006/0275237 and 2004/0175347, both filed by Bissett, et al.

In some instances, the compositions used according to the present method include from 0.001% to 40% (e.g., from 1% to 30%, or from 2% to 20%) of one or more particulate materials and/or cosmetic powders to provide acute look and/or feel benefits. These particulates can, for instance, be platelet shaped, spherical, elongated or needle-shaped, or irregularly shaped; surface coated or uncoated (e.g., hydrophobically coated); porous or non-porous; charged or uncharged; and can be added to the current compositions as a powder or as a pre-dispersion. For example, pigmentary-grade metal oxide particles (e.g., having an average primary particle size greater than 100 nm or from 100 nm to 500 nm) may optionally be included to provide an appearance benefit. Some nonlimiting examples of particulate materials for use herein are described in U.S. Publications Nos. 2012/0021027, 2010/0074928, 2010/0003205, 2010/0003293 and 2013/0243835.

In another example, the compositions used in accordance with the present method may include powders in the form of spherical particles, provide an acute look and/or feel benefit. Spherical particle powders tend to improve the speed that the product appears to absorb into the skin, which helps provide increased control over product application (e.g., less likely to get into the eye and cause irritation). Spherical particle powders herein have a median particle size of 2 μm to 40 μm, (e.g., 3 μm to 25 μm or even 5 μm to 15 μm). Spherical particle powders can also increase the smooth feeling of the product film on the skin. Accordingly, it may be desirable to select spherical particles that have no tackiness and a rubber hardness (as measured by Durometer A defined in JIS K 6253) in the range of 10 to 90, (e.g., 20 to 80 or even from 25 to 75). In a particularly suitable example, the composition includes 2% to 20% (e.g., 4% to 12%) spherical silicone elastomer particles or spherical starch particles. The amount of silicone elastomer powder in the composition is determined based on the particulate material being in neat form (i.e., not swollen in solvent). Some nonlimiting examples of spherical particle powders are described in co-pending U.S. Ser. Nos. 14/596,360 and 14/596,374, filed by Jansen, et al., on Jan. 14, 2015.

### Methods

### Visual Perception Method

This method provides a way to quantitatively evaluate the change in appearance of periorbital dyschromia using a Visual Perception Scale (“VPS”). The visual grading described herein is conducted by trained graders on captured images of the test subjects, but the method may also be readily adapted for use by consumers in self-diagnosing periorbital dyschromia and/or by in vivo examination of the periorbital region of a person by another. For example, it may be desirable to train beauty consultants who interact with consumers in a retail environment to classify periorbital dyschromia. Comparisons of baseline images collected at week 0 versus subsequent time point images are performed. The degree of change is scored using a −4 to +4 Magnitude Scale as shown below in Table 1. Negative numbers indicate that the periorbital dyschromia appeared better at baseline, while positive numbers reflect an improvement of the subject's appearance relative to baseline. The area of the periorbital region graded encompasses the area of the eye socket generally under the eye, extending from the inner corner of the eye, along the cheek bone and around to the outer corner of the eye, inclusive of the lateral orbital rim. The area of the periorbital region graded in this method does not include the area directly below the lower eyelid (as demarcated by the lower eyelashes), the upper eyelid or the upper eye socket. Features considered by the graders include: 1) the relative appearance of the darkness of the discoloration of the periorbital dyschromia compared to the surrounding skin tone; 2) the amount of affected area, footprint or pattern of the periorbital dyschromia; and 3) the appearance of the pigmentation hues involved in the discoloration and their intensity.

TABLE 1
Magnitude Scale

| Grade | Anchor | Description |
|---|---|---|
| +4 | Outstandingly | Significant improvement in contrast, area and/or |
| | Improved | intensity throughout the graded area; outstanding |
| | | improvement is immediately seen. |
| +3 | Obviously | Readily seen improvements in contrast, area and/or |
| | Improved | intensity are obvious almost instantly. |
| +2 | Visibly Improved | Visible improvement in contrast, area and/or |
| | | intensity is able to be seen within a few seconds. |
| +1 | Perceptibly | Improvement in contrast, area and/or intensity are |
| | Improved | perceived after careful study. |
| 0 | Neutral/No | No changes, or equivalent positive and negative changes, |
| | Difference | in the graded area.* |
| | | *Images should not be over scrutinized; images |
| | | requiring more than 30 seconds of study to identify |
| | | a change should be scored as having “zero” |
| | | change. |
| −1 | Perceptibly | Worsening in contrast, area and/or intensity are |
| | Worsened | perceived after careful study. |
| −2 | Visibly Worsened | Visible worsening in contrast, area and/or intensity |
| | | is able to be seen within a few seconds. |
| −3 | Obviously | Readily seen worsening in contrast, area and/or |
| | Worsened | intensity are obvious almost instantly. |
| −4 | Outstandingly | Significant worsening in contrast, area and/or |
| | Worsened | intensity throughout the graded area; outstanding |
| | | improvement is immediately seen. |

### Blood Perfusion Method

Blood perfusion is generally recognized as the process of delivering blood to a capillary bed in biological tissue. Blood vessels and blood in the capillary beds of the periorbital region may be visible through the relatively thin periorbital skin. Thus, when less blood is visible in and around the capillary beds of the periorbital skin, there is a corresponding improvement in the appearance of periorbital dyschromia. The Blood Perfusion Method provides a suitable method of measuring the change in the amount of blood present in the capillary beds of periorbital skin.

The Blood Perfusion Method uses a blood perfusion imager (e.g., PeriCam™ PSI brand imager or equivalent), which is based on Laser Speckle Contrast Analysis (“LASCA”) technology, in conjunction with PIMsoft™ brand dedicated application software or equivalent to visualize tissue blood perfusion in real-time. Test subjects are comfortably seated within 10 to 25 cm of the imager and instructed to close their eyes. Three images (i.e., perfusion, intensity and a standard color image) of the test subject's face are captured and recorded by the imager in accordance with the manufacturer's instructions. Using the dedicated application software, the periorbital regions of the test subject are masked (i.e., designated as regions of interest) to obtain the perfusion measurement in a periorbital region of interest. Masking is described in more detail below in the Imaging Method.

### Contrast Ratio Method

Herein, “contrast ratio” refers to the opacity of a composition (i.e., the ability of the composition to reduce or prevent light transmission), determined after the composition is drawn onto an opacity chart (Form N2A, Leneta Company of Manwah, N.J. or the equivalent thereof). Contrast Ratio is measured using a spectrophotometer with settings selected to exclude specular reflection. The composition is applied to the top of the opacity chart and then is drawn into a film having a thickness of approximately 25 microns using a film applicator (e.g., as commercially available from BYK Gardner of Columbia, Md., or the equivalent thereof). The film is allowed to dry for 2 hours under conditions of 22° C.+/−1° C., 1 atm. Using a spectrophotometer with the settings selected to exclude specular reflection, the Y tristimulus value (i.e., the XYZ color space of the film) of the product film is measured and recorded. The Y tristimulus value is measured in three different areas of the product film over the black section of the opacity chart, and also in three different areas of the product film over the white section of the opacity chart.

The contrast ratio is calculated as the mathematical average of the three Y tristimulus values over the black areas, divided by the mathematical average of the three Y tristimulus values over the white areas, times 100:

$$Contrast Ratio = \frac{average ( Yblack )}{average ( Ywhite )} \times 100$$

### Imaging Method

This method provides a means for capturing a reproducible and analyzable image for determining L*a*b* values and for VPS testing. Any suitable image capture device along with imaging software and other associated ancillary equipment (e.g., computer and lights) may be used. A particularly suitable imaging system is the Visia-CR® brand imaging system, available from Canfield Scientific, New Jersey. The Visia® brand imaging system incorporates a Canon® brand EOS-1Ds Mk III SLR camera, which includes a CMOS sensor and provides 21.1 Mega pixel resolution (14-bit A/D converter).

Images may be collected under different lighting modalities using standard light, UV, cross-polarization, parallel-polarization or a combination of these. For example, the values and ranges described herein are reported using a (D65/2) light source. One skilled in the art will appreciate that these values can be reported at a wide range of different illuminations (D50, D75, Illuminant A, F2, F7, F11, TL84, etc. or 2 or 10 degree observer) according to well-known conversion methods, and when such conversions occur, the color values will typically change accordingly. In other words, even though the actual limits and/or ranges may change based on the conditions under which the image is captured, similar relationships among the values and ranges will still be seen. For example, if the camera has lower spectral sensitivity in the red channel than the camera described herein, the R channel response may be lower and the corresponding L*a*b* color values will be different, which in this case may result in lower a* values and/or higher b* values. Accordingly, different camera sensitivities, lightings and relevant exposures are contemplated, and the actual limits and/or ranges disclosed herein may vary according to the particular circumstances in which the image is captured without departing from the scope of the systems and/or methods described herein.

In preparation for image capture, test subjects are required to wash their faces and wait for at least 15 minutes to let their face dry. The hair of the subject is covered with a hairnet and the head and shoulders of the subject are covered with a black cloth. All jewelry that can be seen in an image area of interest is removed. The subject is positioned such that the subject's chin is resting comfortably on the chin rest of the imaging system, and a front image of the face (as opposed to a left-side or right-side image) can be suitably captured by the image capture device. After the subject is positioned, one or more images are captured (e.g., between 1 and 24, 2 and 20 or even between 3 and 15) with the subject's eyes open. It can be important to ensure that the subject's eyes are open when the image is captured, otherwise the closed upper eyelid may cause an inaccurate pigmentation reading. The captured image(s) are processed by converting the raw image to a .jpg file format.

Next, the .jpg format image is analyzed by a computer with suitable image analysis software. In some instances, it may be desirable to analyze only a portion of the image (e.g., Zone 1, 2 and/or 3 of the periorbital region). The portion of the image to be analyzed may be “masked” using image editing software such as Photoshop® or ImageJ® brand software. The masked region can then be isolated and analyzed as a separate image. It is to be appreciated that the image need not necessarily be masked for suitable analysis, and in some instances the entire image may be analyzed. In some instances, it may be desirable to reduce the size of the image, mask and/or region of interest by several pixels (e.g., between 5 and 15 pixels) around the outer edge of the image where some shadowing may occur.

The RGB values in the image, which are device dependent are converted to L*a*b* values. The L*a*b* values can be calculated using a suitable RGB conversion tool at D65 Illuminant and 2 degree observer (i.e., D65/2) (e.g., software installed on the computer or a suitable conversion tool found online). The conversion from RGB values to L*a*b* values can be performed on the entire image, a portion thereof or on one or more individual pixels. The resulting L*a*b* values may be averaged to provide average values for the image, mask or region of interest.

In some instances, the pixels may be analyzed individually and each pixel classified as corresponding to a particular type of periorbital dyschromia based on one or more of the L*a*b* values. When analyzed individually, the pixels may be analyzed according to their distribution across the different types of periorbital dyschromia. Since color may be perceived as being relative, depending on, for example, which instruments and/or imaging system is used, it can be important to color correct the masked region for each subject using a suitable color correction technique (e.g., according to International Color Consortium standards and practices), which helps make the color determination by the system less instrument specific. In some instances, it may be desirable to normalize the color in a region of interest (e.g., a masked region) to the basal skin tone of a nearby region (e.g., cheek). For example, the basal skin tone of the cheek may be obtained by masking a region of interest in the cheek and converting the RGB values in the masked region to L*a*b* values as described above. The resulting basal skin tone values for the cheek may then be subtracted from the corresponding values in the region of interest to provide normalized values. Color normalization may be performed on the entire region of interest (e.g., an average value for the ROI) or on a pixel by pixel basis for some or all of the pixels in the ROI, which may be 200,000 or more pixels.

### EXAMPLES

### Example 1—Formulation Examples

Table 2 shows five exemplary oil-in-water emulsion cosmetic compositions for use according to the present method. Compositions A to E may be prepared as follows. Combine the water phase ingredients in a suitable vessel and heat to 75° C. In a separate suitable vessel, combine the oil phase ingredients and heat to 75° C. Add the oil phase to the water phase and mill the resulting emulsion (e.g., with a TEKMAR™ T-25 or equivalent). Add the thickener to the emulsion and cool to 45° C. while stirring. At 45° C., add the remaining ingredients. Cool the product with stirring to 30° C. and pour into suitable containers.

TABLE 2

| | A | B | C | D | E |
|---|---|---|---|---|---|
| Water Phase: | | | | | |
| Water | qs | qs | qs | qs | qs |
| Glycerin | 3.0 | 5.0 | 7.0 | 10.0 | 15.0 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2.0 | 0.5 | 3.5 | 3.0 | 5.0 |
| D-panthenol | 0.5 | 0.1 | 1.0 | 0.5 | 1.5 |
| Sodium Hydroxide | 0.001 | 0.002 | 0.001 | 0.001 | 0.001 |
| Benzyl alcohol | 0.25 | 0.25 | 0.25 | 0.25 | 0.25 |
| FD&C Red #40 | — | — | — | 0.0005 | — |
| Cucurbita pepo extract⁴ | 1.0 | 15.0 | 0.1 | 5.0 | 2.0 |
| Palmitoyl-pentapeptide¹ | 0.0002 | — | — | — | 0.0003 |
| N-acetyl glucosamine | 2.0 | — | 2.0 | — | 5.0 |
| Oil Phase: | | | | | |
| Isohexadecane | 3.0 | 3.0 | 3.0 | 4.0 | 3.0 |
| Isopropyl Isostearate | 1.0 | 0.5 | 1.3 | 1.5 | 1.3 |
| Sucrose polyester | 0.7 | — | 0.7 | 1.0 | 0.7 |
| Octinoxate | — | — | — | — | 6.0 |
| Avobenzone | — | — | — | 2.0 | 0.5 |
| Ethylhexyl | — | — | — | — | 0.5 |
| methoxycrylene | | | | | |
| Homosalate | — | — | — | 4.0 | — |
| Octisalate | — | — | — | 4.0 | — |
| Octocrylene | — | — | — | 2.0 | — |
| Phytosterol | — | — | — | 0.1 | — |
| Cetyl alcohol | 0.4 | 0.3 | 1.0 | 0.5 | 0.4 |
| Stearyl alcohol | 0.5 | 0.35 | 1.0 | 0.6 | 0.5 |
| Behenyl alcohol | 0.4 | 0.3 | 1.0 | 0.5 | 0.4 |
| PEG-100 stearate | 0.1 | 0.1 | 0.1 | 0.2 | 0.1 |
| Stearic Acid | 0.1 | 0.05 | 0.1 | 0.2 | 0.1 |
| Cetearyl glucoside | 0.1 | 0.1 | 0.1 | 0.25 | 0.1 |
| Thickener: | | | | | |
| Polyacrylamide/C13-14 | 1.5 | — | 2.0 | 2.5 | 2.0 |
| isoparaffin/laureth-7 | | | | | |
| Sodium | — | 3.0 | — | — | — |
| acrylate/sodium | | | | | |
| acryloyldimethyl | | | | | |
| taurate | | | | | |
| copolymer/isohexadecane/ | | | | | |
| polysorbate 80 | | | | | |
| Additional Ingredients: | | | | | |
| KTZ Interfine ™ Gold² | 2.5 | — | 0.3 | — | 0.5 |
| KTZ Interfine ™ Red² | — | 1.0 | — | — | 0.5 |
| Tapioca Starch | — | 5.0 | — | 2.0 | 0.5 |
| Dry Flo TS³ | 8.0 | — | 1.5 | — | — |
| Dimethicone/dimethiconol | — | 1.0 | 2.0 | 0.5 | 2.0 |
| Fragrance | — | 0.1 | 0.1 | 0.1 | 0.1 |
| Polymethylsilsequioxane | — | — | 0.25 | — | 1.0 |
| Nylon-12 | — | 0.5 | — | — | — |
| Total: | 100% | 100% | 100% | 100% | 100% |

¹Palmitoyl-lysine-threonine-threonine-lysine-serine available from Sederma (France)
²Titanium dioxide coated mica available from Kobo Products Inc.
³Tapioca starch and polymethylsilsesquioxane from Akzo Nobel
⁴OCALINE PF from Soliance, France

Table 3 shows five exemplary silicone-in-water emulsion cosmetic compositions for use according to the present method. Compositions F to J may be prepared as follows. In a suitable vessel, combine the water phase ingredients and mix until uniform. In a separate suitable container, combine the silicone/oil phase ingredients and mix until uniform. Add half the thickener and then the silicone/oil phase to the water phase and mill the resulting emulsion (e.g., with a Tekmar™ T-25). Add the remainder of the thickener and then the remaining ingredients to the emulsion while stirring. Once the composition is uniform, pour the product into suitable containers.

TABLE 3

| | F | G | H | I | J |
|---|---|---|---|---|---|
| Water Phase: | | | | | |
| Water | qs | qs | qs | qs | qs |
| Glycerin | 3.0 | 5.0 | 7.0 | 10.0 | 15.0 |
| Disodium EDTA | 0.1 | 0.1 | 0.05 | 0.1 | 0.1 |
| Niacinamide | 2.0 | 0.5 | 3.5 | 3.0 | 5.0 |
| D-panthenol | 0.5 | 0.1 | 1.0 | 0.5 | 1.5 |
| Cucurbita pepo extract⁹ | 1.0 | 2.0 | 0.1 | 10.5 | 1.5 |
| FD&C Yellow #10 | — | — | — | — | 0.0004 |
| Palmitoyl- | 0.0002 | — | — | — | 0.0003 |
| pentapeptide¹ | | | | | |
| N-acetyl glucosamine | 2.0 | — | 2.0 | — | 5.0 |
| Silicone/Oil Phase: | | | | | |
| Cyclomethicone D5 | 10.0 | 5.0 | 5.0 | 10.0 | 7.5 |
| Dow Corning ® 9040 | — | 10.0 | 5.0 | 5.0 | 7.5 |
| silicone elastomer² | | | | | |
| KSG-15AP silicone | 5.0 | — | 5.0 | 5.0 | 7.5 |
| Elastomer³ | | | | | |
| Dimethione/ | — | 2.0 | 2.0 | 1.0 | 2.0 |
| dimethiconol | | | | | |
| Dimethicone 50 csk | 1.0 | — | — | — | — |
| Laureth-4 | 0.2 | 0.2 | 0.3 | 0.2 | 0.2 |
| Vitamin E Acetate | — | 0.5 | — | 0.1 | — |
| Thickener: | | | | | |
| Polyacrylamide/C13- | 2.5 | 2.5 | 3.0 | — | — |
| 14 | | | | | |
| isoparaffin/laureth-7 | | | | | |
| Sodium | — | — | — | 3.0 | — |
| acrylate/sodium | | | | | |
| acryloyldimethyl | | | | | |
| taurate | | | | | |
| copolymer/isohexadecane/ | | | | | |
| polysorbate 80 | | | | | |
| Acrylates/C10-30 | — | — | — | — | 0.5 |
| alkyl acrylates | | | | | |
| crosspolymer | | | | | |
| Additional Ingredients: | | | | | |
| KSP 100⁴ | 6.0 | 1.5 | — | — | — |
| KTZ Interval ™ | — | 0.35 | — | 1.0 | 0.8 |
| Green⁵ | | | | | |
| Prestige Silk ™ | — | — | 1.5 | — | — |
| Blue⁶ | | | | | |
| Cosmica ™ Orange⁷ | — | — | — | 0.1 | — |
| Dry Flo TS⁸ | — | 1.5 | 8.0 | — | — |
| Fragrance | — | 0.1 | 0.1 | 0.1 | 0.1 |
| Triethanolamine | — | — | — | — | 0.6 |
| PTFE | — | 0.5 | — | — | — |
| Polymethylsilsequioxane | — | 0.5 | 1.0 | — | — |
| Polyethylene | — | 0.5 | — | — | 1.0 |
| Total: | 100% | 100% | 100% | 100% | 100% |

¹Palmitoyl-lysine-threonine-threonine-lysine-serine available from Sederma (France)
²A silicone elastomer dispersion from Dow Corning Corp.
³A silicone elastomer dispersion from Shin Etsu
⁴Vinyl dimethicone/methicone silsesquioxane crosspolymer from Shinetsu
⁵Titanium dioxide coated mica from Kobo Products Inc.
⁶Titanium dioxide and tin oxide coated mica from Eckart.
⁷Iron oxide coated mica from Engelhard Corporation.
⁸Tapioca starch and polymethylsilsesquioxane from Akzo Nobel
⁹OCALINE PF from Soliance, France

Table 4 shows two exemplary water-in-silicone emulsion cosmetic compositions for use according to the present method. Compositions K and L may be prepared as follows. In a suitable vessel, blend the Phase A components with a suitable mixer until all of the components are dissolved. Blend Phase B components in suitable vessel and mix until uniform. Add Phase A slowly to Phase B with mixing and continue mixing until uniform. Mill the resulting product for about 5 minutes using an appropriate mill (e.g., TEKMAR T-25). Next, add Phase C while stirring the product. Continue mixing until the product is uniform, and pour the product into suitable containers.

TABLE 4

| | K | L |
|---|---|---|
| Phase A | | |
| Water | q.s. | q.s. |
| Allantoin | 0.2 | 0.2 |
| Disodium EDTA | 0.1 | 0.1 |
| Ethyl paraben | 0.2 | 0.2 |
| Propyl paraben | 0.1 | 0.1 |
| BHT | 0.015 | 0.015 |
| D-panthenol | 1.0 | 0.5 |
| Glycerin | 7.5 | 13.0 |
| Niacinamide | 2.0 | 3.5 |
| Palmitoyl-pentapeptide¹ | — | 0.0003 |
| Benzyl alcohol | 0.2500 | 0.2500 |
| Green tea extract | 1.0 | 0.1 |
| Cucurbita pepo extract⁶ | 1.0 | 2.2 |
| Sodium metabisulfite | 0.1 | 0.01 |
| Phase B | | |
| Cyclopentasiloxane | 15.0000 | 15.0000 |
| C12-C15 alkyl benzoate | 1.5 | — |
| Vitamin E acetate | 0.5 | 0.1 |
| Retinyl propionate | 0.15 | — |
| Phytosterol | 0.1 | — |
| KSG-21 silicone elastomer² | 4.0 | 4.0 |
| Dow Corning ® 9040 silicone | 15.0 | 15.0 |
| elastomer³ | | |
| Abil ™ EM-97 dimethicone | 0.5 | — |
| copolyol⁴ | | |
| Polymethylsilsesquioxane | 2.5 | 0.5 |
| Fragrance | — | 0.1 |
| Phase C | | |
| KTZ Interval ™ Red-11S2⁵ | — | 0.3 |

¹Palmitoyl-lysine-threonine-threonine-lysine-serine available from Sederma (France)
²KSG-21 is an emulsifying silicone elastomer available from Shin Etsu
³A silicone elastomer dispersion from Dow Corning Corp
⁴Abil EM-97 available from Goldschmidt Chemical Corporation
⁵Silane surface treated titanium dioxide coated mica from Kobo Products Inc.
⁶OCALINE PF from Soliance, France.

Table 5 shows examples of personal care compositions for use in the present method. The compositions may be prepared by first combining the water phase ingredients in a container and mixing while heating to ˜75° C. until uniform. Meanwhile, the ingredients of part 1 of the oil phase are weighed into a separate container and mixed while heating to ˜75° C. until uniform. Once both respective phases are uniform, part 1 of the oil phase is added to the water phase. The resulting mixture is subjected to high shear mixing (e.g., Flacktek Speedmixer, or rotor-stator mill) and then cooled while stirring. When the temperature, reaches ˜60° C., the thickener is then added while continuing to stir. Finally, when the batch reaches ˜50° C., the Oil Phase Part 2 is added ingredients are added individually as cooling continues. At ˜40° C., the active (i.e., pumpkin seed extract) is added while stirring. Once all ingredients are in the formulation and the temperature is ˜40° C., the resulting mixture is again subjected to high shear mixing, and then the product is poured into suitable containers.

TABLE 5

| | M | N | O | P | Q | R |
|---|---|---|---|---|---|---|
| Water Phase: | | | | | | |
| Water | 84.26 | 84.22 | 84.17 | 84.02 | 83.77 | 83.27 |
| Glycerin | 5.0 | 5.0 | 5.0 | 5.0 | 5.0 | 5.0 |
| Disodium | 0.1 | 0.1 | 0.1 | 0.1 | 0.1 | 0.1 |
| EDTA | | | | | | |
| Oil Phase | | | | | | |
| Part 1: | | | | | | |
| Isohexadecane | 3.0 | 3.0 | 3.0 | 3.0 | 3.0 | 3.0 |
| Isopropyl | 1.33 | 1.33 | 1.33 | 1.33 | 1.33 | 1.33 |
| Isostearate | | | | | | |
| Polymethyl- | 0.25 | 0.25 | 0.25 | 0.25 | 0.25 | 0.25 |
| silsesquioxane | | | | | | |
| Cetearyl | 0.20 | 0.20 | 0.20 | 0.20 | 0.20 | 0.20 |
| Glucoside, | | | | | | |
| Cetearyl | | | | | | |
| Alcohol | | | | | | |
| Behenyl | 0.40 | 0.40 | 0.40 | 0.40 | 0.40 | 0.40 |
| Alcohol | | | | | | |
| Ethylparaben | 0.20 | 0.20 | 0.20 | 0.20 | 0.20 | 0.20 |
| Propylparaben | 0.10 | 0.10 | 0.10 | 0.10 | 0.10 | 0.10 |
| Cetyl Alcohol | 0.32 | 0.32 | 0.32 | 0.32 | 0.32 | 0.32 |
| Stearyl Alcohol | 0.48 | 0.48 | 0.48 | 0.48 | 0.48 | 0.48 |
| PEG-100 | 0.10 | 0.10 | 0.10 | 0.10 | 0.10 | 0.10 |
| Stearate | | | | | | |
| Thickener: | | | | | | |
| Sepigel 305¹ | 2.00 | 2.00 | 2.00 | 2.00 | 2.00 | 2.00 |
| Oil Phase | | | | | | |
| Part 2: | | | | | | |
| Benzyl Alcohol | 0.25 | 0.25 | 0.25 | 0.25 | 0.25 | 0.25 |
| DC 1503² | 2.00 | 2.00 | 2.00 | 2.00 | 2.00 | 2.00 |
| Active: | | | | | | |
| Cucurbita | .01% | 12% | 0.1% | 6% | 2% | 5% |
| pepo extract³ | | | | | | |
| Total: | 100% | 100% | 100% | 100% | 100% | 100% |

¹Polyacrylamide, C13-14 isoparaffin, and laureth-7, from Seppic, France.
²Dimethicone and Dimethiconol from Dow Corning, Inc., Midland, MI.
³OCALINE PF from Soliance, France.

### Example 2: In Vivo Study (VPS, Blood Perfusion and Imaging)

This example demonstrates the ability of the present method to improve the appearance of Type II and Type III periorbital dyschromia. Twenty-five Caucasian female test subjects aged 20 to 60 were enrolled in a nine-week, split-face, round-robin design study to evaluate the ability of a pumpkin seed extract to improve the appearance of Type II and Type III periorbital dyschromia. The oil-in-water emulsion of Example R from Table 5 was evaluated in this study.

During the study, the under-eye portion of the periorbital region (i.e., the shaded area 400 in FIG. 4A) on the left side of the test subject's face was treated with the test composition, and the under-eye portion of the periorbital region on the right side of the test subject's face was treated with a vehicle control (i.e., the same composition as the test composition except without pumpkin seed extract). The test subjects were instructed to use cleansing cloths and a facial moisturizer, which were provided to them, twice a day. The test subjects were also instructed to refrain from using any eye treatment products during the course of the study and to avoid excessive UV exposure that could result in facial sunburn or tanning. The test subjects were permitted to use their normal makeup products (e.g., foundation, blush, eye and lip liners) five minutes after application of the under-eye compositions, but were asked not to switch brands. The test subjects applied the control and test compositions twice a day; once in the morning and once in the evening at least 30 minutes before going to bed. Approximately 0.04 g or 40-50 μl of each composition was applied to the periorbital skin under the appropriate eye. Images of the test subjects and blood perfusion data were collected at weeks 0 (baseline), 2, 4 and 8 for use in the Visual Perception Scale, Imaging and Blood Perfusion Methods described above. The baseline values were determined at the start of the test (week 0). The control values shown in the Tables below are an average across all test subjects.

The results of the in vivo study are illustrated below in Tables 6, 7 and 8 for test subjects exhibiting Type II periorbital dyschromia and in Tables 9, 10 and 11 for test subjects exhibiting Type III periorbital dyschromia. The results illustrated in Tables 6-11 are averages of mean values. For each paired comparison, each endpoint was analyzed using a mixed model, which included subject (random effect), treatment effect, and fixed effects (side of the face and baseline). In this test, a one-sided p-value was used to compare the efficacy of the treatment as compared to control. P-values of 0.2 or less and 0.8 or more are considered statistically significant, and p-values of less than 0.3 but greater than 0.2 and less than 0.8 but greater than 0.7 are considered statistically trending. From the results it can be seen that the pumpkin seed extract provided an improvement in the appearance of periorbital dyschromia.

Table 6 shows the change in VPS relative to the baseline value for treatment of Type II periorbital dyschromia with the test composition and the vehicle control.

TABLE 6
Type II - VPS

| | | Test | |
|---|---|---|---|
| | Control | Composition | p-Value |
| Week 2 | −0.11 | 0.79 | 0.04 |
| Week 4 | 0.39 | 1.03 | 0.08 |
| Week 8 | −0.31 | 0.66 | 0.10 |

Table 7 shows the change in blood perfusion value relative to the baseline value for treatment of Type II periorbital dyschromia with the test composition and the vehicle control.

TABLE 7
Type II - Blood Perfusion

| | | Test | |
|---|---|---|---|
| Week | Control | Product | p-value |
| Week 2 | −9.48 | −3.34 | 0.72 |
| Week 4 | −5.5 | −8.27 | 0.37 |
| Week 8 | −11.2 | −2.55 | 0.77 |

Table 8 shows the change in imaging values (i.e., L* value, a* value and b* value) relative to the baseline value for treatment of Type II periorbital dyschromia with the test composition and the vehicle control

TABLE 8
Type II - Imaging Values

| | | | Test | p- |
|---|---|---|---|---|
| Endpoint | Week | Control | Product | value |
| L* | Week 2 | 0.99 | 1.15 | 0.35 |
| L* | Week 4 | 1.58 | 1.35 | 0.78 |
| L* | Week 8 | 1.04 | 1.32 | 0.21 |
| a* | Week 2 | −0.27 | −0.28 | 0.49 |
| a* | Week 4 | −0.45 | −0.41 | 0.59 |
| a* | Week 8 | −0.37 | −0.26 | 0.72 |
| b* | Week 2 | 0.42 | 0.36 | 0.62 |
| b* | Week 4 | 0.48 | 0.38 | 0.69 |
| b* | Week 8 | 0.32 | 0.33 | 0.48 |

Table 9 shows the change in VPS scores relative to the baseline value for treatment of Type III periorbital dyschromia with the test composition and the vehicle control.

TABLE 9
Type III - VPS

| | | Test | p- |
|---|---|---|---|
| Week | Control | Product | Value |
| 2 | 0.63 | −0.29 | 0.94 |
| 4 | −0.66 | −0.18 | 0.19 |
| 8 | 0.76 | 0.14 | 0.79 |

Table 10 shows the change in blood perfusion value relative to the baseline value for treatment of Type III periorbital dyschromia with the test composition and the vehicle control.

TABLE 10
Type III - Blood Perfusion

| | | Test | p- |
|---|---|---|---|
| Week | Control | Product | value |
| 2 | 2.59 | 5.35 | 0.55 |
| 4 | −16.8 | −0.6 | 0.88 |
| 8 | 10.91 | −0.52 | 0.24 |

Table 11 shows the change in imaging values (i.e., L* value, a* value and b* value) relative to the baseline value for treatment of Type III periorbital dyschromia with the test composition and the vehicle control.

TABLE 11
Type III - Imaging Values

| | | | Test | p- |
|---|---|---|---|---|
| Endpoint | Week | Control | Product | value |
| L* | 2 | 1.1 | 0.62 | 0.83 |
| L* | 4 | 0.35 | 1.03 | 0.03 |
| L* | 8 | 1.3 | 1.12 | 0.65 |
| a* | 2 | 0.14 | 0.27 | 0.64 |
| a* | 4 | 0.37 | 0.14 | 0.14 |
| a* | 8 | −0.3 | −0.09 | 0.77 |
| b* | 2 | 0.27 | 0.01 | 0.75 |
| b* | 4 | 0.18 | 0.25 | 0.40 |
| b* | 8 | 0.48 | 0.43 | 0.55 |

### Example 3: In Vitro Study (B16—Melanin Assay)

This example demonstrates the inability of pumpkin seed extract to inhibit melanin synthesis. It is believed that an overabundance of melanin is a key contributor to the appearance of Type I periorbital dyschromia, but not for Type II periorbital dyschromia. Thus, treating Type I periorbital dyschromia with pumpkin seed extract should not provide any improvement in its appearance, as demonstrated by the lack of melanin inhibition activity in a conventional B16 assay. This is important because it shows that a “one size fits all” approach may not be the best way to treat all types of periorbital dyschromia. For example, a composition that utilizes pumpkin seed extract may not improve the appearance of Type I periorbital dyschromia.

In this example, a commercially available B16-F1 mouse melanoma cell line from American Tissue Culture Collection, Virginia, USA was employed in a conventional melanin synthesis inhibition assay. The cell culture medium used in the assay is 500 mL of Dulbecco's Modified Eagle's Medium (DMEM), 50 mL Fetal Bovine Serum (FBS), and 5 mL of penicillin-streptomycin liquid. B16-F1 cells that are cultured in this medium and grown to greater than 90% confluency will synthesize melanin. While not intending to be bound by any theory, it is hypothesized that the melanin synthesis is stimulated by the culture medium and/or stress induced by growth to a high confluency. The DMEM and FBS can be obtained from American Tissue Culture Collection and the penicillin-streptomycin liquid can be obtained from Invitrogen, Inc., California, USA. Equipment used in the assay include a CO₂ incubator, such as a Forma Series Model 3110 by Therma Scientific, Massachusetts, USA; a hemocytometer, such as a Bright Line model by Hauser Scientific, Pennsylvania, USA; and a UV-Visible Spectrum Plate Reader, such as a SpectraMax250 from Molecular Devices, California, USA.

Day 0: To begin the assay, the cell culture medium is heated to 37° C. and 29 mL of the medium is placed into a T-150 flask. Approximately 1×10⁶ of B16-F1 passage 1 mouse cells are added to the T-150 flask and incubated for 3 days at 37° C., 5% CO₂, 90% relative humidity, until ˜80% confluency.

Day 3: The cells from the T-150 flask are trypsinized, and the concentration of cells is determined using the hemacytometer. Initiate a 96 well plate with 2,500 cells per well in 100 μL of cell culture medium. Incubate the plate at 37° C., 5% CO2, 90% relative humidity for 2 days until at least 20% to 40% confluent.

Day 5: Remove the cell culture medium from the plate and replace with fresh culture medium (100 uL per well). Add 1 uL of test compound diluted in a water solvent. Multiple dilution ratios may be tested in order to generate a dose response curve, wherein preferably three wells are treated with each dilution ratio. Positive and negative controls may include wells having the cell culture medium, B16-F1 cells, and the solvent (negative control), and wells comprising the cell culture medium, B16-F1 cells and a known melanin inhibitor (e.g., deoxyarbutin or kojic acid).

Day 7: Cells should have a confluency greater than ˜90%. If not, this data point is not used. Add 100 uL of a 0.75% sodium hydroxide solution to each well. Read the 96 well plate using the UV-Vis Plate Reader at 410 nm to optically measure the amount of melanin produced between wells that are treated with the pumpkin seed extract and control wells that are not. Wells in which melanin is produced appear brownish in color. Wells in which little melanin is produced appear clear to light purple in color. Percentage of melanin synthesis inhibition is calculated by the following equation:

$$\frac{100 - [ OD 410 Test Compound - OD 410 Control \#2 ] \times 100}{( OD 410 Control \#1 - O D 410 Control \#2 )}$$

Where OD410 is the Optical Density at 410 nm as measured by the UV-Vis Spectrum Plate Reader.

When Control #3 is used, the formula for percentage melanin synthesis inhibition is:

$$\frac{100 - [ OD 410 Test Compound - OD 410 Control \#3 ] \times 100}{( OD 410 Control \#1 - O D 410 Control \#3 )}$$

The concentration of test agent needed to provide the IC 50 is recorded.

Table 12 shows the concentration of each composition needed to provide the IC 50. The positive controls used in this example are deoxyarbutin and kojic acid, both of which are well-known inhibitors of melanin synthesis. As shown in Table 12, the concentration of the test composition required to obtain IC 50 was much higher than either the deoxyarbutin or the kojic acid, suggesting that the pumpkin seed extract tested in this example is a poor inhibitor of melanin synthesis.

TABLE 12
B16 (IC 50)

| | Concentration |
|---|---|
| | Needed for IC 50 |
| Composition | (v/v) |
| Deoxyarbutin | 0.008 |
| Kojic Acid | 0.01 |
| Cucurbita pepo extract¹ | 0.4 |

¹OCALINE PF from Soliance, France.

A. A method of improving the appearance of periorbital dyschromia, comprising:
   i. identifying a target portion of skin in a periorbital region of a person that exhibits Type II or Type III periorbital dyschromia; and
   ii. applying a personal care composition to the target portion of skin during a treatment period, the personal care composition comprising an effective amount of an extract of Cucurbita pepo, a dermatologically acceptable carrier, and a viscosity of from about 50,000 to about 200,000 cps, and the treatment period is sufficient for the extract of Cucurbita pepo to improve the appearance of the periorbital dyschromia.
B. The method of paragraph A, wherein the improvement in appearance corresponds to a positive change on a visual perception scale (VPS).
C. The method of paragraph A or B, wherein the improvement in appearance corresponds to a decrease in blood perfusion.
D. The method of any preceding paragraph, wherein the improvement in appearance corresponds to an increase in L* value, a decrease in a* value, an increase in b* value or a combination of these.
E. The method of any preceding paragraph, wherein the personal care composition is applied locally to a periorbital region of a user.
F. The method of any preceding paragraph, wherein the personal care composition does not worsen the appearance of Type I periorbital dyschromia.
G. The method of any preceding paragraph, further comprising classifying the Type II or Type III periorbital dyschromia using a method selected from visual evaluation, blood perfusion, image analysis, histological analysis, biomarker analysis, gene expression signature analysis, gene expression theme analysis, and combinations of these.
H. The method of any preceding paragraph, further comprising consulting a trained beauty consultant or using a diagnostic device to classify the Type II or Type III periorbital dyschromia.
I. The method of any preceding paragraph, wherein the personal care composition comprises from about 0.0001% to about 15% by weight of an extract of Cucurbita pepo.
J. A method of improving the appearance of periorbital dyschromia, comprising:
   i. identifying a target portion of skin exhibiting periorbital dyschromia;
   ii. applying a first personal care composition to the target portion of skin during a treatment period, wherein the first personal care composition comprises from about 0.0001% to about 15% by weight of the composition of an extract of Cucurbita pepo and a dermatologically acceptable carrier; and
   iii. applying a second personal care composition to the target portion of skin, the second personal care composition comprising a skin care agent and a dermatologically acceptable carrier,
wherein the treatment period is sufficient amount for the extract of Cucurbita pepo to improve the appearance of the periorbital dyschromia.
   K. The method of paragraph J, wherein the first composition is applied locally to the target portion of skin and the second composition is applied generally to facial skin.
   L. The method of paragraph J or K, wherein the second composition is applied after the first composition is applied.
   M. The method of any preceding paragraph, wherein the target portion of skin exhibits Type II periorbital dyschromia.
   N. The method of any preceding paragraph, wherein the extract is present at an amount of from 0.05% to 5%.
   O. The method of any preceding paragraph, wherein the improvement in appearance corresponds to a positive change in VPS score.
   P. The method of any preceding paragraph, wherein the improvement in appearance corresponds to a decrease in blood perfusion.
   Q. The method of any preceding paragraph, wherein the personal care composition does not worsen the appearance of Type I periorbital dyschromia.
   R. The method of any preceding paragraph, wherein the improvement in appearance corresponds to an increase in L* value, a decrease in a* value, an increase in b* value, or a combination of these.
   S. The method of any preceding paragraph, further comprising classifying the Type II or Type III periorbital dyschromia using a method selected from visual evaluation, blood perfusion, image analysis, histological analysis, biomarker analysis, gene expression signature analysis, gene expression theme analysis, and combinations of these.
   T. The method of any preceding paragraph, further comprising consulting a trained beauty consultant or using a diagnostic device to classify the Type II or Type III periorbital dyschromia.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving the appearance of periorbital dyschromia, comprising:
a. identifying a target portion of skin in a periorbital region of a person that exhibits Type II or Type III periorbital dyschromia; and
b. applying a personal care composition to the target portion of skin during a treatment period, wherein the personal care composition comprises an effective amount of an extract of Cucurbita pepo, a dermatologically acceptable carrier, and a viscosity of from about 50,000 to about 200,000 cps, and the treatment period is sufficient for the extract of Cucurbita pepo to improve the appearance of the periorbital dyschromia.

2. The method of claim 1, wherein the improvement in appearance corresponds to a positive change on a visual perception scale (VPS).

3. The method of claim 1, wherein the improvement in appearance corresponds to a decrease in blood perfusion.

4. The method of claim 1, wherein the improvement in appearance corresponds to an increase in L* value, a decrease in a* value, an increase in b* value, or a combination of these.

5. The method of claim 1, wherein the personal care composition is applied locally to a periorbital region of a user.

6. The method of claim 1, wherein the personal care composition does not worsen the appearance of Type I periorbital dyschromia.

7. The method of claim 1, further comprising classifying the Type II or Type III periorbital dyschromia using a method selected from visual evaluation, blood perfusion, image analysis, histological analysis, biomarker analysis, gene expression signature analysis, gene expression theme analysis, and combinations of these.

8. The method of claim 1, further comprising consulting a trained beauty consultant or using a diagnostic device to classify the Type II or Type III periorbital dyschromia.

9. The method of claim 1, wherein the personal care composition comprises from about 0.0001% to about 15% by weight of an extract of Cucurbita pepo.

10. A method of improving the appearance of periorbital dyschromia, comprising:
a. identifying a target portion of skin exhibiting periorbital dyschromia;
b. identifying a target portion of skin exhibiting periorbital dyschromia;
c. applying a first personal care composition to the target portion of skin during a treatment period, wherein the first personal care composition comprises from about 0.0001% to about 15% by weight of the composition of an extract of Cucurbita pepo and a dermatologically acceptable carrier; and
d. applying a second personal care composition to the target portion of skin, the second personal care composition comprising a skin care agent and a dermatologically acceptable carrier,
wherein the treatment period is sufficient for the extract of Cucurbita pepo to improve the appearance of the periorbital dyschromia.

11. The method of claim 10, wherein the first composition is applied locally to the target portion of skin and the second composition is applied generally.

12. The method of claim 10, wherein the second composition is applied after the first composition is applied.

13. The method of claim 10, wherein the target portion of skin exhibits Type II periorbital dyschromia.

14. The method of claim 10, wherein the extract is present at an amount of from 0.05% to 5%.

15. The method of claim 10, wherein the improvement in appearance corresponds to a positive change in VPS score.

16. The method of claim 10, wherein the improvement in appearance corresponds to a decrease in blood perfusion.

17. The method of claim 10, wherein the personal care composition does not worsen the appearance of Type I periorbital dyschromia.

18. The method of claim 10, wherein the improvement in appearance corresponds to an increase in L* value, a decrease in a* value, an increase in b* value, or a combination of these.

19. The method of claim 10, further comprising classifying the Type II or Type III periorbital dyschromia using a method selected from visual evaluation, blood perfusion, image analysis, histological analysis, biomarker analysis, gene expression signature analysis, gene expression theme analysis, and combinations of these.

20. The method of claim 10, further comprising consulting a trained beauty consultant or using a diagnostic device to classify the Type II or Type III periorbital dyschromia.
